(19) United States
(12) Patent Application Publication
(10) Pub. No.: US 2003/0023284 A1
(43) Pub. Date: Jan. 30, 2003

(54) Method and apparatus for the in-vivo treatment of pathogens

(76) Inventors: Vladimir Gartstein, Cincinnati, OH (US); Faiz Feisal Sherman, West Chester, OH (US); Ying Xu, Wakefield, MA (US); Milan Marcel Jevtitch, Cincinnati, OH (US); Donald Carroll Roe, West Chester, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/078,043

(22) Filed: Feb. 19, 2002

Related U.S. Application Data
(60) Provisional application No. 60/269,927, filed on Feb. 20, 2001.

(51) Int. Cl. A61N 5/06; A61B 18/18; A61N 1/00; A61B 19/00

(52) U.S. Cl. 607/88; 607/94; 606/2; 128/898

(57) ABSTRACT

A method and apparatus for the meaningful suppression of the growth potential of a pathogen in-vivo, the apparatus including an electromagnetic radiation source capable of providing broad-spectrum electromagnetic radiation, wherein the broad-spectrum electromagnetic radiation has wavelengths of from about 190 nm to about 1200 nm, the broad-spectrum electromagnetic radiation having an intensity sufficient to achieve meaningful suppression in the growth potential of the pathogen in-vivo and wherein at least part of the apparatus is adapted for placement proximate to the in-vivo location of the pathogen.

### CROSS REFERENCE

This is application claims priority to Provisional Application Serial No. 60/269,927, filed Feb. 20, 2001.

### TECHNICAL FIELD

The invention relates to devices adapted to treat pathogen-based infections, such as bacterial infections, for example, acute otitis media, streptococcus infections, Staphylococcus aureus; viral infections, for example, the common cold, HIV, and prion caused diseases, such as, kuru, Bovine Spongiform Encephalopathy (popularly known as ‘mad cow disease’) etc. The invention also relates to a method for treating pathogen-based infections.

### BACKGROUND OF THE INVENTION

Current means of treating pathogen based infections of various types include chemical and pharmaceutical treatments, such as antibiotic treatment, radiation, and surgery. Antibiotic treatment has a significant disadvantage in that frequent usage increases the likelihood of developing bacterial strains resistant to future antibiotic treatments, jeopardizing the future health of the individual and society. Surgical treatment has the disadvantage of being highly invasive and, therefore, impractical in many cases. While radiation treatment often involves inadvertent damage to the host tissues or organs.

In many circumstances a healthy organism's own immune system is capable of dealing with an infection caused by a pathogen if given sufficient time. However, the time for the organism's own immune system to deal with the infection can result in longer duration of illness, permanent side effects, economic loss, and generally be detrimental to the well-being of the organism. It is also possible that opportunistic infections by other pathogens can occur while the organism is still responding to the primary pathogenic illness. Use of chemical and pharmaceutical treatments to aid in the organism's immune system is one possible solution, however potential side effects, as well as the possibility of resistant pathogens are issues of increasing concern.

Additionally, in immunocompromised organisms, their own immune systems are less able or incapable of dealing with pathogen caused infection. Any such pathogen caused infection may be difficult to eradicate using conventional chemical and pharmaceutical treatments. Immunocompromised organisms include, for example, those infected by HIV, those undergoing chemotherapy, transplant recipients, or cancer patients receiving immunosuppressive medications.

Consequently, the need remains for a method that is capable of eliminating and/or controlling pathogens in-vivo and/or providing in-vivo assistance to an organisms immune response to a wide rage of pathogens, is convenient and simple to use, and does not have the likelihood of developing pathogen strains resistant to future treatments by the same or similar methods.

### SUMMARY OF THE INVENTION

Surprisingly, a way has been found to eliminate pathogens in-vivo and alternatively provide in-vivo assistance to an organism's immune response to a wide range of pathogens, which is convenient and simple to use, and does not have the likelihood of developing pathogen strains resistant to future treatments by the same or similar methods.

In accordance with a first aspect of the present invention, an apparatus for the meaningful suppression of the growth potential of a pathogen in-vivo is provided. The apparatus comprises an electromagnetic radiation source capable of providing broad-spectrum electromagnetic radiation, wherein the broad-spectrum electromagnetic radiation has wavelengths of from about 190 nm to about 1200 nm, the broad-spectrum electromagnetic radiation having an intensity sufficient to achieve meaningful suppression in the growth potential of the pathogen in-vivo and wherein at least part of the apparatus is preferably adapted for placement proximate to the in-vivo location of the pathogen.

In accordance with a second aspect of the present invention, a method for achieving the meaningful suppression of the growth potential of a pathogen in a living organism is provided. The method comprises administering a broad-spectrum electromagnetic radiation to the living organism to the locus of the pathogen in the living organism and wherein the broad-spectrum electromagnetic radiation has wavelengths of from about 190 nm to about 1200 nm, the broad-spectrum electromagnetic radiation having an intensity sufficient to achieve meaningful suppression in the growth potential of the pathogen in-vivo.

In accordance with a third aspect of the present invention, a method for aiding the immune response of a living organism to a pathogen by temporarily suppressing the pathogen is provided. The method comprises administering a broad-spectrum electromagnetic radiation to the living organism at or near the locus of the pathogen in the living organism, wherein the broad-spectrum electromagnetic radiation has a wavelength of from about 190 nm to about 1200 nm, and an intensity from about 0.01 J/cm² to about 1 J/cm², and the pathogen is suppressed by increasing the time for the pathogen to double in population.

In accordance with a fourth aspect of the present invention, a stimulating the immune system of an organism is provided. The method comprises administering a broad-spectrum electromagnetic radiation to the living organism wherein the broad-spectrum electromagnetic radiation has wavelengths of from about 190 nm to about 1200 nm, the broad-spectrum electromagnetic radiation having an intensity sufficient to stimulate the immune system of the organism. It is preferred that the broad-spectrum electromagnetic radiation is applied to a specific locus of said living organism, such as trauma and pathogen. Examples of trauma include, cuts, abrasions, lesions, burns, damage caused by chemotherapy and the like.

These and other aspects, features and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified. All patents, articles, documents, and other materials cited are, in relevant part, incorporated herein by reference; the citation of any document is not to be construed as an admission that it is prior art with respect to the present invention.

### BRIEF DESCRIPTION OF THE DRAWINGS

While the specification concludes with claims particularly pointing out and distinctly claiming the present invention, it is believed the present invention will be better understood from the following description in which:

FIG. 1 is a view of one embodiment of the present invention, namely a hand-held apparatus.

FIG. 2 is a cut away view of the apparatus of FIG. 1.

FIG. 3 is a view of another embodiment of the present invention, namely an apparatus into which the organism or part thereof is placed.

FIG. 4 is a view of another embodiment of the present invention, namely a device that is adapted for delivering the broad-spectrum electromagnetic radiation to the locus of a pathogen that is reached through a natural entrance to the interior of an organism.

It should be understood that the drawings are not necessarily to scale and that the embodiments are some tines illustrated by graphic symbols, phantom lines, diagrammatic representations and fragmentary views. In certain instances, details which are not necessary for an understanding of the present invention or which render other details difficult to understand may have been omitted. It should be understood, of course, that the invention is not limited to the particular embodiments illustrated herein.

### DETAILED DESCRIPTION OF THE INVENTION

Definitions

As used herein, the term “meaningful suppression of the growth potential of a pathogen” means that there is either a permanent or temporary interruption in the reproduction cycle of the pathogen. This includes increasing the pathogen's “proliferation time”, which is herein defined as the time required for a pathogen's population to double in number. Preferably, the increase in proliferation time representing a meaningful suppression of the pathogen's growth potential is typically a doubling of the proliferation time, with even longer time increases, such as tripling the proliferation time or even longer time increases within the scope of the present invention. However, any time that increases the proliferation time sufficient to aid an organism's immune system response is suitable. The population doubling time will vary depending upon the pathogen, the organism, and the locus of the pathogen. Alternatively, the “meaningful suppression of the growth potential of a pathogen” can be measured as a reduction in the concentration of viable organisms. Preferably the treatment achieves at least about a 1 log reduction in the population of the pathogen, more preferably at least about a 2 log reduction, even more preferably at least about a 6 log reduction. Finally, in life threatening circumstances either because of the pathogen, or because of the condition of the organism the permanent interruption in the reproduction cycle of the pathogen includes the elimination or near total elimination of the pathogen from the organism. Examples of the former would be when the pathogen was, HIV, Methicillin-resistant Staphylococcus aureus, or Hepatitis C and the like. Example of the later would be organisms undergoing chemotherapy, organ transplant recipients who's immune system is suppressed to prevent rejection of transplanted organ, organisms with weakened or failed immune system and the like.

As used herein, the term “temporarily suppressing the pathogen” includes not only meaningful suppression of the growth potential of a pathogen but also rendering the pathogen more susceptible to an organism's immune system, or more susceptible to chemical and/or pharmaceutical treatments.

As used herein, the term “organism” includes any multi cellular living plant or animal. This includes both domestic and wild plants and animals. The definition does not include within the scope of its meaning biologically derived compositions, such as whole blood, blood products such as plasma, milk, etc. One type of organisms are those possessing an immune system.

As used herein, the term “animal” includes all living multi cellular animals, both wild and domestic varieties, as well as terrestrial and aquatic animals, and invertebrates and vertebrates. One type of animal are those possessing an immune system. Examples of animals meeting this definition include, but are not limited to, mammals, reptiles, amphibians, birds, insects and the like. The term “animal” also includes, but is not limited to, farm animals, such as, cows, chickens, sheep, goats, llamas, pigs, crocodiles, alligators, rabbits, minks, deer, moose, salmon, oysters, emu, ostriches, ducks, quail, pheasants, partridges, bees, turkeys, geese, horses, etc; domestic animals, such as, dogs, cats, frogs, turtles, skinks, ants, beetles, goldfish, parrots, canaries, mice, hamsters, rats, etc; zoo or wild animals, such as, lions, buzzards, snipe, albatross, rhinos, dolphins, pandas, lizards, wombats, kangaroos, camels (both single and double humped varieties), seals, etc; and primates, such as, humans, monkeys, lemurs, gibbons, gorillas, and baboons.

As used herein, the term “plant” includes all living multi cellular plants, both wild and domestic varieties. Examples of plants meeting this definition include, but are not limited to: flowering plants, (e.g. tulips, daisies, roses), conifers, aquatic plants such as seaweed, commercial plants such as coffee, wheat, corn, barley, potatoes, grapes, apples, and cut or harvested flowers, such as those available commercially.

As used herein, the term “electromagnetic radiation source” means either a single source, or multiple sources, capable of providing a broad-spectrum electromagnetic radiation, which is defined in more detail hereinafter. The electromagnetic radiation source may be a single source providing the entire spectrum required for the broad-spectrum electromagnetic radiation. Alternatively, the electromagnetic radiation source may include a number of sources, each providing at least a portion of the entire spectrum required, such that the combination of sources provides the required broad-spectrum electromagnetic radiation.

As used herein, the term “broad-spectrum electromagnetic radiation” is intended to mean either a continuous or discontinuous band of electromagnetic radiation which includes at least a portion of electromagnetic radiation from the visible spectrum and at least a portion of electromagnetic radiation from ultraviolet B and/or ultraviolet C spectra. The term “continuous band” means that the all wavelengths from the lowest to the highest are included in the broad-spectrum electromagnetic radiation. On the other hand, the term “discontinuous band” means that not all of the wavelengths from the lowest to the highest are included in the broad-spectrum electromagnetic radiation. For example, part of the red visible spectrum may be omitted from the broad-spectrum electromagnetic radiation. Alternatively, the visible light range may be approximated by two or more “colors”, or wavelength ranges, of light, such as a combination of red, green, and blue light. In certain preferred embodiments, the broad-spectrum electromagnetic radiation has an infrared component, a visible component, an ultra violet-A component, an ultra violet-B component and an ultra violet-C component. For example, this could be a continuous band from about 190 nm to about 1200 nm or alternatively it could be a discontinuous band with a an infrared component, a visible component, an ultra violet-A component, an ultra violet-B component and an ultra violet-C component. In certain embodiments, the broad-spectrum electromagnetic radiation includes at least a visible component, and component with a wavelength from about 190 nm to less than or equal to 300 nm.

As used herein, the term “intensity” means the strength or power of the broad-spectrum electromagnetic radiation at the locus or in-vivo location of the pathogen. Typically, the intensity of the broad-spectrum electromagnetic radiation is sufficient to achieve meaningful suppression in the growth potential of the pathogen in-vivo.

As used herein, the term in-vivo means in the interior or inside of a living organism, such as in the inner ear, lung, stomach, mouth, etc, or on the exterior or outside of a living organism, such as on the skin, fur, claw or shell of an organism.

As used herein, the term “pathogen” includes biological substances capable of proliferation that causes a disease or an illness in an organism. This includes, but is not limited to viruses, bacteria, pyrogens, toxins, fungi, protozoa, prions and combinations thereof. The term pathogen includes within its meaning not only those pathogens which are organism specific, but also those which are found in more than one organism or more than one species. That is, for example, were the pathogen causes similar diseases in different organisms, causes different diseases in different organisms, or only causes a disease in one organism, but resides in one or more other organisms causing no harm and in effect acting as a reservoir of pathogens from which infection of susceptible organisms can result.

As used herein, the term “natural entrance” means a naturally occurring point of access to at least a portion of the interior of an organism. Some exemplary natural entrances include, but are not limited to, ear, nostrils, anus, mouth, urethra, vagina, eye and tear duct.

As used herein, the term “other than through a natural entrance” means other than naturally occurring point of access to at least a portion of the interior portions of an organism, most likely as a result of human intervention. Some exemplary natural entrances include, but are not limited to, incision, stoma, trachea tube, myringotomy tube and combinations thereof.

As used herein, the term “acute tissue effect” means some temporary effects in the organism at the locus of the pathogen. Typically, such acute tissue effect includes, but is not limited to erythema, scaling, swelling, dimerisation of DNA, protein degradation and/or inflammation.

As used herein, the term “chronic effects” means some long term and/or permanent effects in the organism at the locus of the pathogen. Typically, such chronic effect includes, but is not limited to, cleavage of DNA.

As used herein, the term “proximate” means some position that is in close physical proximity, to the locus of the pathogen in the organism. For example, for pathogens in the inner ear, placement proximate would mean insertion into the ear in reasonable proximity to the pathogen in the inner ear, such as near the tympanic membrane.

As used herein, the terms “remotely located” or “location remote” means an operator and/or programmer of the apparatus is in some fashion distant from the physical location of the apparatus. Typically, this means they are not in physical contact with the apparatus. The term includes, relatively short distances between the apparatus and operator/programmer. The term also includes, relatively long distances the apparatus and operator/programmer. Examples of the former include, the apparatus in a room and the operator/programmer on the other side of the same room, in another room, in another building or even in another suburb. Examples of the former include, the apparatus in one location and the operator/programmer in a different city, in a different state, in a different country, in a different continent. The terms “remotely located” and “location remote” also includes when the apparatus is located on a means of transportation, such as a plane, train, ship, space shuttle or the like, and the operator/programmer is not present on the means of transportation. Additionally, the term “remotely located” and “location remote” includes when the apparatus and/or the operator/programmer is in some remote geographical location, such as an oil-drilling platform, a space station, and the like.

1) Apparatus

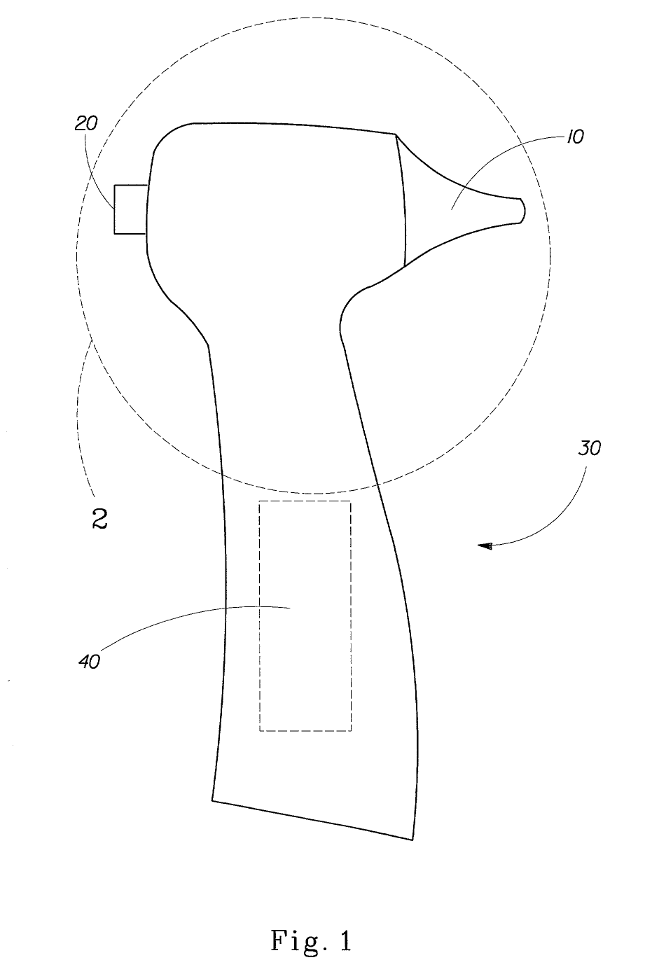

FIG. 1 shows a side view of a hand held apparatus 30 according to one embodiment of the present invention. The apparatus 30 includes a power source 40 which is enclosed in the device as shown by broken lines, an on/off switch 20, and part of the apparatus which is adapted for placement proximate to the in-vivo location of the pathogen 10.

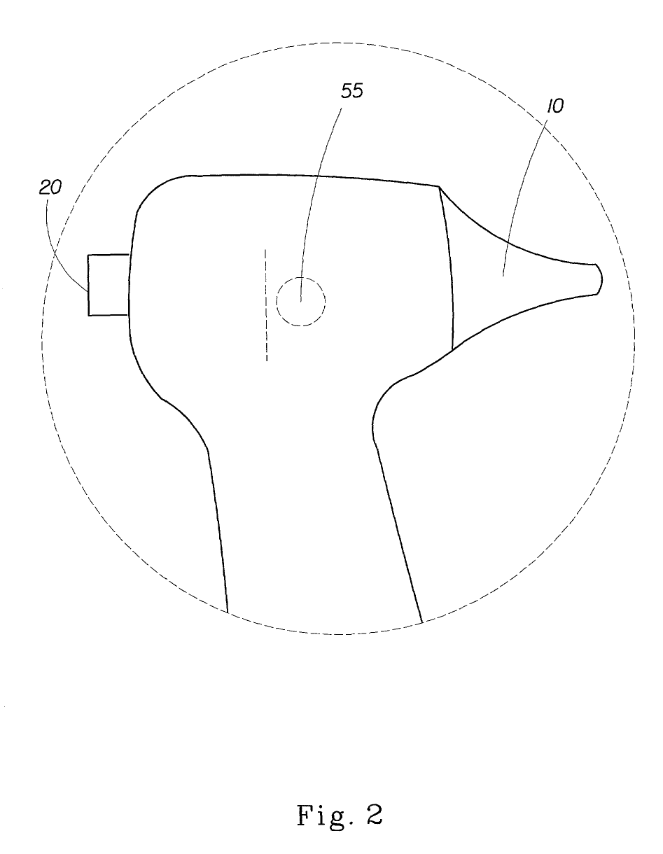

FIG. 2 shows a cutaway view along the hand held apparatus of FIG. 1. The apparatus 30 shows an on/off switch 20, an electromagnetic radiation source capable of providing broad-spectrum electromagnetic radiation 55 and part of the apparatus that is adapted for placement proximate to the in-vivo location of the pathogen 10.

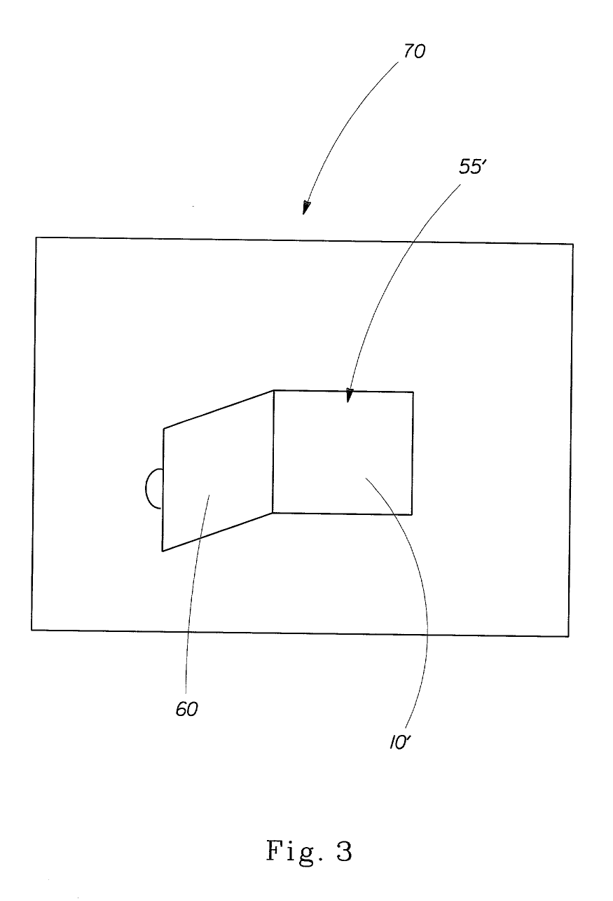

FIG. 3 shows a side view of an apparatus 70 according to one embodiment of the present invention. The apparatus 70 includes a door 60, an electromagnetic radiation source capable of providing broad-spectrum electromagnetic radiation 55′ and part of the apparatus which is adapted for placement proximate to the in-vivo location of the pathogen 10′. In this case, 10′ is a chamber into which the organism or part thereof is placed.

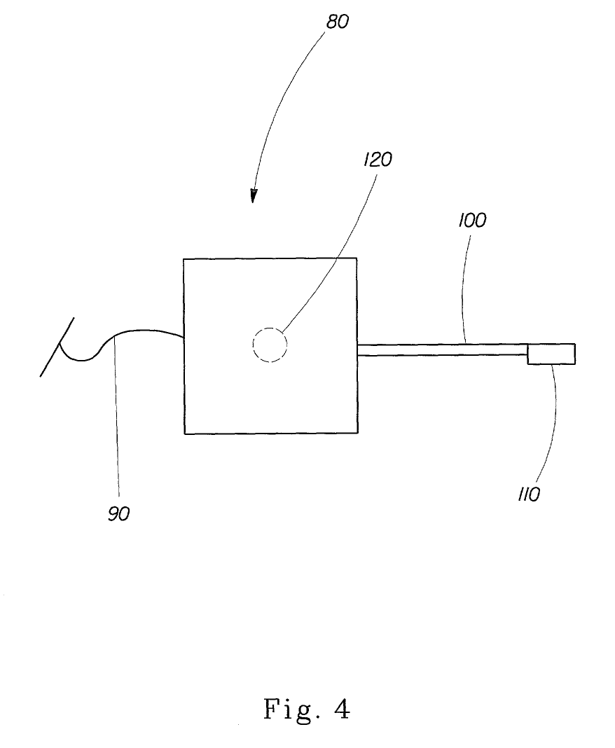

FIG. 4 shows a side view of an apparatus 80 according to one embodiment of the present invention. The apparatus 80 includes a connection to a power source 90, an electromagnetic radiation source 120, part of the apparatus which is adapted for placement proximate to the in-vivo location of the pathogen 110 and a fiber optic cable 100 for linking the electromagnetic radiation source 120 to the part adapted for placement which is adapted for placement proximate to the in-vivo location of the pathogen 110.

In the first embodiment of the present invention provides an apparatus for the meaningful suppression of the growth potential of a pathogen in-vivo. The apparatus may contain a variety of optional features as discussed hereinafter, however, the apparatus according to the present invention typically comprises an electromagnetic radiation source capable of providing broad-spectrum electromagnetic radiation, wherein the broad-spectrum electromagnetic radiation has wavelengths of from about 190 nm to about 1200 nm, the broad-spectrum electromagnetic radiation having an intensity sufficient to achieve meaningful suppression in the growth potential of the pathogen in-vivo and wherein at least part of the apparatus is preferably adapted for placement proximate to the in-vivo location of the pathogen

In one embodiment of the present invention the broad-spectrum electromagnetic radiation comprises pulsed broad-spectrum electromagnetic radiation. The broad-spectrum electromagnetic radiation may be pulsed by electrical, mechanical and/or electro-mechanical means. For example, the broad-spectrum electromagnetic radiation could be pulsed by turning the source of the broad-spectrum electromagnetic radiation on and off, by varying the intensity of the broad-spectrum electromagnetic radiation, or by interposing something such as a shutter or the like between the source of the broad-spectrum electromagnetic radiation and the in-vivo location of the pathogen.

In one embodiment of the present invention the number of times the electromagnetic radiation is preferably pulsed is at least about one pulse, more preferably about 3 pulses, even more preferably about 20 pulses. Similarly the apparatus pulses the electromagnetic radiation preferably pulsed a number of times no greater than about 1000 pulses.

In one embodiment of the present invention each pulse of the electromagnetic radiation has duration of preferably about 1 microsecond, more preferably about 1 millisecond, even more preferably about 100 milliseconds, even more preferably still about 500 milliseconds.

In the apparatus of the present invention, the intensity is that which is sufficient to achieve meaningful suppression in the growth potential of the pathogen in-vivo. Intensity is measured as energy per unit of area of the entire spectrum of electromagnetic energy, typically Joules/cm², or J/cm². Preferably the intensity of the electromagnetic radiation is at least about 0.01 J/cm², more preferably at least about 0.05 J/cm², even more preferably at least about 0.1 J/cm², even more preferably still at least about 0.2 J/cm². Similarly, the intensity of the electromagnetic radiation is preferably no greater than about 1 J/cm², more preferably no greater than about 0.75 J/cm², even more preferably no greater than about 0.6 J/cm², even more preferably still at least about 0.5 J/cm². In any event, intensity selected should be based on the pathogen, the in-vivo location of the pathogen, and the organism in which the in-vivo location is.

Energy to be delivered, as well as pulse frequency and duration, may be varied based on type of pathogen, location on/in organism and engineering considerations. A non-limiting example is 0.05 J/cm², (50 mJ/cm²) via 25 pulses within a two second time span, with 10-microsecond pulse duration.

In one preferred embodiment of the present invention the apparatus of the present invention supplies a broad-spectrum electromagnetic radiation of an intensity that minimizes acute tissue effects at the in-vivo location of the pathogen. Acute tissue effects on the organism in or near the locus of the pathogen are temporary in nature. These typically include, erythema, redness, swelling, scaling and/or inflammation. It is also preferred that the apparatus of the present invention supplies a broad-spectrum electromagnetic radiation of an intensity that minimizes, or more preferably does not produce chronic effects at the in-vivo location of the pathogen. Chronic effects on the organism in or near the locus of the pathogen are either permanent or long term in nature. These typically include, dimerization of DNA, cleavage of DNA, and/or protein degradation. In any event the risk of possible acute or chronic effects should be considered in light of the pathogen to be treated. For example, mild erythema is likely undesirable for treatment of acute otitis media, whereas even possible chronic side effects would be acceptable for treatment of life threatening illnesses such as HIV, Ebola, or even Creutzfeldt-Jakob disease.

In one embodiment of the present invention the electromagnetic radiation source may comprise a single source. That is, a single bulb or the like capable of providing the broad-spectrum electromagnetic radiation required for the apparatus of the present invention. Alternatively, electromagnetic radiation source may comprise multiple sources. That is, combination of coherent and/or incoherent light sources, such as lasers, bulbs or the like capable of providing the broad-spectrum electromagnetic radiation required for the apparatus of the present invention. Suitable sources of electromagnetic radiation include, but are not limited to, halogen lamps, xenon, lamps, halogen enhanced UV lamps, xenon flash lamps, mercury xenon lamps, deuterium lamps, vacuum UV lamps, mercury lamps, lasers and combinations thereof. Exemplary lasers, or sources of coherent light, include argon, krypton, neon, and xenon lasers. Commercially available examples include, but not limited to: Miniature series of halogen lamps (spectra 380 nm to 770 nm) available from Welch Allyn, Skaneateles Falls, N.Y., USA; Sub Miniature series of halogen enhanced UV lamps (spectra 240 nm to 770 nm) also available from Welch Allyn, Skaneateles Falls, N.Y., USA; L2000 Series xenon lamps (spectra 185 nm to 2000 nm) available from available from Xenon Corporation, Bridgewater, N.J., USA; L2000, L4000, L6000 and L7000 Series xenon flash lamps (spectra 160 nm to 2000 nm) also available from available from Xenon Corporation, Bridgewater, N.J., USA; L2000 Series mercury xenon lamps (spectra 185 nm to 2000 nm) also available from available from Xenon Corporation, Bridgewater, N.J., USA; L2D2 Series deuterium lamps (spectra 185 nm to 400 nm) available from available from Xenon Corporation, Bridgewater, N.J., USA; Miniature series of xenon lamps (spectra 185 nm to 2000 nm) available from THHC Lighting, City of Industry, Calif., USA; Miniature series of xelogen lamps (spectra 380 nm to 2000 nm) also available from THHC Lighting, City of Industry, Calif., USA; Miniature series of halogen lamps (spectra 380 nm to 770 nm) also available from THHC Lighting, City of Industry, Calif., USA; deuterium lamps (spectra 185 nm to 400 nm) available from Cathodeon Ltd, Cambridge UK; xenon lamps (spectra 185 nm to 2000 nm) also available from Cathodeon Ltd, Cambridge UK; mercury lamps (spectra 185 nm to 2000 nm) also available from Cathodeon Ltd, Cambridge UK; RSL2100 xenon flash lamps (spectra 200 nm to 1000+ nm) available from Perkins Elmer, Santa Clara USA; and 1100 Series (spectra 120 nm to 1000+ nm) also available from Perkins Elmer, Santa Clara USA.

In any event, if one or multiple sources are used precautions should be taken to ensure that the desired spectrum remains constant for the duration of the treatment. For example, prolonged continuous use of a lamp may result in the generation of heat that will cause the spectrum of the broad-spectrum electromagnetic radiation generated by the bulb to shift, reducing the control of the treatment. This can be solved in many different ways including pulsing to prevent heat build up sufficient to shift the spectrum, or by cooling of the bulb to prevent heat build up. In any event whatever solution is used, pulsing, cooling or the like, it is preferable that the spectrum remains constant for the duration of the treatment. One preferred approach is to minimize the duty cycle (i.e., the firing duration divided by the time span between the initiation of each flash, or pulse, of the lamp, and commonly expressed as a percentage) to prevent overheating of the bulb. Preferably, the duty cycle is less than about 1% for passively cooled bulbs. The duty cycle may be somewhat higher if the lamp is actively cooled (i.e., forced convection via a fan, etc.). However, while less preferred, it is still within the scope of the present invention that the spectrum of the broad-spectrum electromagnetic radiation may change or shift, during treatment. It is to be understood that if the spectra does shift during treatment that any spectra resulting which is used in connection with treatment falls within the definition of “broad-spectrum electromagnetic radiation” as given herein.

In one embodiment of the present invention the broad-spectrum electromagnetic radiation is a continuous band. In an alternative embodiment of the present invention the broad-spectrum electromagnetic radiation comprises a multiplicity of discrete bands (i.e., relatively narrower wavelength distributions) of electromagnetic radiation. Two or more of the discrete bands may at least partially overlap one another (i.e., share some common wavelengths) or may be completely separate.

In one embodiment of the present invention the apparatus comprises at least one filter to remove wavelengths. Alternatively, multiple filters can be used. In embodiments of the apparatus comprising multiple sources of electromagnetic radiation one filter may be used to cover all of the sources or multiple filters, such as one each for each electromagnetic radiation source.

Typically the broad-spectrum electromagnetic radiation is either a continuous or discontinuous band of electromagnetic radiation which includes at least a portion of electromagnetic radiation from the visible spectrum and at least a portion of electromagnetic radiation from ultraviolet B and/or ultraviolet C spectra. In one preferred embodiments of the present invention, the broad-spectrum electromagnetic radiation includes at least a visible component, and component with a wavelength from about 190 nm to less than or equal to 300 nm, more preferably at least a visible component, and component with a wavelength from about 190 nm to less than or equal to 250 nm.

In one preferred embodiment of the present invention the broad-spectrum electromagnetic radiation excludes wavelengths that are absorbed by the in-vivo location. For example, the band of visible red light could be excluded from a broad-spectrum electromagnetic radiation, especially when the locus to which it is applied already shows symptoms of erythema, that is, redness of tissue. While not wanting to be limited by theory, it is believed that elimination of the red portion will reduce the amount of broad-spectrum electromagnetic radiation absorbed by the organism and create or not increase any erythema in the organism. Alternatively, for treatment involving the exterior or outside of an organism those frequencies that would be absorbed by the, skin, scales, feather etc, may also similarly, be excluded from the broad-spectrum electromagnetic radiation.

In one preferred embodiment of the present invention the apparatus of the present invention is capable of providing either a continuous band or discrete bands of electromagnetic radiation. The operator of the apparatus is able to select between continuous band and discrete bands. It is even more preferred that the operator be able to select which frequencies or bands of electromagnetic radiation may be omitted from the broad-spectrum electromagnetic radiation.

It is to be understood that for different pathogens, different intensities of broad-spectrum electromagnetic radiation (i.e., energy to be delivered) are selected as is frequency and duration of pulses for pulsed broad-spectrum electromagnetic radiation. Additionally, the proportion of the total delivered energy provided via a given wavelength band may be varied based on the organism and the properties of the locus to be treated without the organism.

In one preferred embodiment of the present invention the apparatus comprises a controller. The controller manages the duration and intensity of said electromagnetic radiation source. The controller may be electrical, mechanical, or electromechanical. Alternatively, the controller may be an algorithm that is specific for different pathogens and different locations. For example, the operator selects the pathogen type, organism, and locus therein and the appropriate pre-programmed algorithm manages the duration and intensity of said electromagnetic radiation source. It is even more preferred that when the apparatus comprises a controller that the electromagnetic radiation is a pulsed broad-spectrum electromagnetic radiation and that the controller manages the pulsing of said electromagnetic radiation.

In another preferred embodiment of the present invention the pulsing, duration and intensity of the broad-spectrum electromagnetic radiation is programmable in to the apparatus by the manufacture of the apparatus, the operator of the apparatus, a third party remotely located from the apparatus and/or combinations thereof. That is, a manufacture could produce an apparatus according to the present invention capable of treating only one type of pathogen, in one specific locus, in one type of organism. Alternatively, a manufacture could produce an apparatus according to the present invention capable of treating a variety of pathogens, in many different loci, in a variety of organisms, for example, one button to select pathogen type, one to select, loci, and another to select organism. The apparatus according the this embodiment of the present invention could also be programmable by the operator, for example selecting which band or bands, intensity, duration, pulsing or non-pulsing and duration of pulses of the broad-spectrum electromagnetic radiation. The operator may be a layperson such as, a homemaker, or may be a trained person such as a doctor, nurse, pharmacist, veterinarian, or the like. The third party remotely located from the apparatus is most likely a doctor, nurse, pharmacist, veterinarian, or the like. For example, the organism may be in some location inaccessible to the third party, such as on an oilrig, in a plane, or in outer space. Alternatively, the organism may be in an accessible location, but it is simpler and/or convenient for the third party to be remotely located from the apparatus. In this embodiment the owner of the apparatus may not be able to select treatment method and after diagnosis of the pathogen and treatment needed, the remote third part programs the apparatus as to the treatment necessary. This is analogous to visiting the doctor and receiving a prescription for medicine. In this case the doctor would make their diagnosis, and provide the treatment information direct to the apparatus. The patient, or their caregiver can then place the apparatus near the locus of the pathogen, for example, up the nose, and activate the device. This would prevent over treatment and allow for the possibility of multiple treatments over a time period. This would also be an advantage for organisms located in remote location. A multiuse apparatus of the present invention could be remotely programmed by a medical practitioner for the necessary treatment regimen.

In another embodiment of the present invention the controller is manageable from a location remote from the apparatus by means of a data link. The controller is operatively connected to the data link, that is the controller receives instructions from some remote third party via the data link. The data link may be of any suitable means for communicating between the remote location and the person who is supplying the information to the controller. Typical examples of suitable data links include, infrared, serial, phone, radiofrequency, optical fiber, coaxial cable, cellular phone (both analogue and digital), satellite, telemetry, and combinations thereof.

In another embodiment of the present invention the apparatus comprises a power source. Typically the power source will be selected based on many factors, including, but not limited to, size of the apparatus, the pathogen to be treated, the locus of pathogen, whether the apparatus will treat multiple pathogens, the power required, etc. Once this has been decided the most suitable power source is selected. Typically, the power source is selected form disposable batteries, fuel cells, mains power, rechargeable batteries, solar power and combinations thereof.

In one embodiment the apparatus of the present invention is a hand held apparatus. That is, the apparatus is portable and can be easily carried. The apparatus illustrated by FIG. 3, is one example of such a hand held apparatus. In another embodiment the apparatus of the present invention is other than hand held. That is, the apparatus may be larger, such as a fixed unit or, movable on wheels or castors, with the part of the apparatus adapted for placement proximate to the in-vivo location of the pathogen being hand held. The apparatus illustrated by FIG. 3, is one example such an apparatus.

In one preferred embodiment of the present invention the apparatus is such that, at least a part of the organism is placed inside the apparatus. For example, this could include, placing the in-vivo location of the pathogen in a chamber in the apparatus, the apparatus includes a bench, seat or examination table on which the organism is placed or places the in-vivo location of the pathogen on. If the organism is an animal, the part of the animal placed inside the apparatus typically could be, for example, the head, torso, arm, leg, foot, wing, beak, flipper, finger, claw, tusks, horn, hooves, tail, hand, toes, and combination thereof of the animal. For example, in an apparatus, which includes an examination table, a person could lie down on the examination table while the broad-spectrum electromagnetic radiation is applied to its leg. Alternatively, the broad-spectrum electromagnetic radiation could be applied to the torso. If the organism were a plant, the part of the plant placed inside the apparatus typically would be a stem, flower, seed, trunk, seed pod, branch, root, fruit, bulb, leaf, tuber, flower, petal and combinations thereof of the plant. Any fruit treated would need to remain attached to the plant during treatment by the apparatus. For example, the apparatus of the present invention may include a vase, into which cut flowers are placed, and the broad-spectrum electromagnetic radiation is delivered to the stems, especially, the cut at the base of the stems, to prolong the life of the cut flowers by retarding and/or eliminating pathogen build up. Another example would be an apparatus, which comprises a dish or similar container, in to which tulip and/or daffodil bulbs are placed for treatment with broad-spectrum electromagnetic radiation.

Typically, in the scope of the present invention pathogen includes any thing that causes a disease, illness, or the like in an organism. Naturally, those pathogens that are most virulent to plants and/or animals are of greatest concern. Typical pathogens include, but are not limited to viruses, bacteria, pyrogens, toxins, fungi, protozoa, prions and combinations thereof.

Each of the above-illustrated type of pathogen treatable by the apparatus of the present invention are now described in some exemplary detail. It is to be understood that the following disclosure of pathogens is not meant to be exhaustive and only illustrative of some pathogens.

Bacteria treatable by the apparatus of the present invention include gram-positive and gram-negative varieties. Naturally, those bacteria that are most virulent to plants and animals are of greatest concern. Some non-limiting examples of bacteria which the apparatus of the present invention achieves meaningful suppression of the growth potential of in-vivo include: Staphylococcus aureus; Staphylococcus epidermidis; Streptococcus; Escherichia coli; Klebsiella pneumoniae; Citrobacter diversus; Enterobacter cloacae; Serratia marcescens; Proteus mirabilis; Proteus vulqaris; Proteus morqanii; Providence species; strains of Haemophilus influenzae; Acinetobacter calcoaceticus; Pseudomonas species; Mycobacterium leprae; Mycobacterium tuberculosis; mycobacterial avium; mycobacterial fortuitium; mycobacterial chelonae; Bacillus stearothermophilus; Bacillus subtilis; Bacillus pumilus; Aspergillus niger; Pseudomonas aeruginosa; Salmonella enteriditis; Burkholderia cepacia (Pseudomonas cepacia); E. coli 0157:H7; Salmonella typhimurium; Listeria monocytogenes; Clostridium sporogenes; Bacillus thuringiensis; Bacillus subtilis var. niger str. globegii; Trichoderma harzianum; Penicillium roquefortii; Sporeforming eukaryote; Penicillium digitatum; Sporeforming eukaryote, Klebsiella terrigena; Pseudomonas aeruginosa; Proteus mirabilis; Salmonella newport ser. Gp C2; Salmonella rubislaw ser. Gp F; Salmonella choleraesuis; Deinococcus radiodurans; Enterococcus faecalis; Deinococcus radiodurans; Enterococcus faecalis; Salmonella choleraesuis; Pseudomonas aeruginosa; Helicobacter pylori; Methicillin-resistant Staphylococcus aureus; Vancomycin-resistant Staphylococcus aureus ; gonorrhea bacteria; penicillin-resistant strain of gonorrhea bacteria; Legionella; Clostridium Novyi type A; Salmonella choleraesuis; Acinetobacter calcoaceticus; tubercle bacillus Mycobacterium tuberculosis; and vancomycin-susceptible and vancomycin-resistant Enterococcus species.

Viruses treatable by the apparatus of the present invention include DNA and RNA viruses as well as enveloped or non-enveloped viruses. Naturally, those viruses that are most virulent to plants and animals are of greatest concern. Viral plant diseases have been known to have a disruptive effect on the cultivation of fruit trees, tobacco, and various vegetables. Insect viral diseases are also of interest because of the insects' ability to transfer viral diseases to humans. Some non-limiting examples of virus which the apparatus of the present invention achieves meaningful suppression of the growth potential of in-vivo include: herpes simplex; herpes zoster (shingles); orofacial herpes zoster; Yellow Fever; Hepatitis A; Hepatitis B; Hepatitis C; Bovine Diarrhea; Poliovirus; reovirus; sindbus virus, encephalomyocarditis virus; vaccinia virus, bacteriophage MS-2; bacteriophage PRD-1; rotavirus; simian rotavirus, Rhinovirus, B19 parvovirus; human papilloma virus; Simian Vacuolating Virus (SV40), Human Immunodeficiency Virus (HIV); Canine parvovirus (CPV); Ross river virus; varicella zoster (chicken pox); the virus causing infections laryngotracheitis in animals; the virus causing infectious bronchitis in animals; the virus causing Newcastle disease, the virus causing hog cholera; the virus causing canine distemper; influenza A virus; Poliovirus LS-c2ab; West Nile virus; cytomegalovirus; Chlamydia pneumonia; and Ebola virus.

A prion is largely, if not entirely, or a self-replicating protein. Examples of some diseases caused by prions treatable by the apparatus of the present invention include, but are not limited to: “Kuru”, an illness originally associated with cannibalism in Papua New Guinea; Bovine Spongiform Encephalopathy (popularly known as “mad cow disease”); Creutzfeldt-Jakob disease; variant Creutzfeldt-Jakob disease; Scapie; and Chronic Wasting Disease, which is a transmissible spongiform disease of North American mule deer and elk;

Fungi treatable by the apparatus of the present invention include are saprophytic or parasitic plants that can cause infections in organisms. This is especially true of immunocompromised organisms are particularly susceptible to fungal infections. In those orgainsms, fungi may cause infections that are difficult to eradicate. Immunocompromised organisms include, for example, those infected by HIV, those undergoing chemotherapy, transplant recipients, or cancer patients receiving immunosuppressive medications. Fungi that attack immunocompromised patients are often called “opportunistic fungi.” These may be opportunistic yeasts, such as species of Candida, Trichosporon, and Cryptococcus.

The presence of fungus may cause various diseases and infections in man including mycotic disease, e.g., pulmonary candidiasis and pulmonary blastomycosis. Certain yeast like organisms, e.g., Cryptococcus neoformans, may cause serious infections of the central nervous system. More commonly known fungal infections in humans and mammals include ringworm, which are fungus infections of hair and nail areas, as well as resistant infections of the skin. Many other fungal infections inflict humans and mammals in the areas of skin, mucous membranes, intestinal tract, vaginal area and lungs.

Some non-limiting examples of fungi treatable by the apparatus of the present invention include: dermatophytes; Trichophyton, such as, Trichophyton rubrum which causes difficult to eradicate nail infections; Microsporum; Epidermophyton; different Candida species; Trichoderma; Cryptococcus; Aspergillus; Zygomyetes; Fusarium; Histoplasmosis; Blastomyces; Coccidioides; Hendersonula toruloidea; tinea capitis; tinea corporis; tinea cruris; fungal candida; and Scopulariopsis brevicaulis.

Additional pathogens treatable by the apparatus of the present invention can be found in: Handbook of Plant Virus Diseases by Dragoljub D. Sutic (Editor), Richard E. Ford (Editor), Malisa T. Tosic; Atlas of Infectious Disease, Volume 12: Fungal Infections (CD-ROM) by Gerald L. Mandell (Editor), Richard D. Diamond (Editor); Plant Pathogenic Bacteria (Current Plant Science and Biotechnology in Agriculture, No 4) by E. L. Civerolo, A. Collmer, R. E. Davis, A. G. Gillaspie (Editor); Microbial Diseases of Fish (Special Publications of the Society for General Microbiology, 9) by Ronald J. Roberts (Editor), Society for General microbial; Veterinary Virology by Frederick A. Murphy (Editor), E. Paul J. Gibbs, Marian C. Horzinek, Michael J. Studdert (Editor); Veterinary Microbiology, by Dwight C. Hirsh (Editor), Yuan Chung Zee (Editor); Topley & Wilson's Microbiology and Microbial Infections, 6-Volume Set; and Mandell, Douglas, and Bennett's Principles and Practice of Infectious Diseases (2 Volume Set) by Gerald L. Mandell (Editor), John E. Bennett (Editor), Raphael Dolin (Editor).

The in-vivo location of the pathogen is an organism. As noted previously, organism includes within its scope both plants and animals. The apparatus may be adapted to apply the broad-spectrum electromagnetic radiation to a specific organism or it could be suitable for use on a wide variety of organisms. The in-vivo location of the pathogen in any particular organism can be the outer surface or exterior of the organism. Non-limiting examples of such outer surfaces include, skin, hair, fur, scales, chitin, shell, nails, claws, hooves, feathers, bark, leaves, flowers, seeds and combinations thereof. Additionally, the in-vivo location of the pathogen can be the interior or inside of the organism. Non-limiting examples of such interiors of organisms include, outer ear, inner ear, throat, vocal cords, mouth, sinus, nostril, eye, tear ducts, bladder, prostate, kidney, urethra, anus, bowel, large intestine, small intestine, trachea, lung, gill, and combinations thereof. These interior in-vivo loci of the pathogens can be typically reached through either a naturally occurring entrance in the organism to its interior, or via other than through a natural entrance to the interior of an organism. Illustrative Examples of the former include, ear, nostrils, anus, mouth, urethra, vagina, eye, tear duct, and combinations thereof. Illustrative examples of the latter include, incision, stoma, trachea tube, myringotomy tube and combinations thereof.

In one preferred embodiment of the present invention, the apparatus is especially adapted for the reduction of pathogens in the auditory system of a mammal. For example, an apparatus could be adapted for the treatment of inner era infections, such as acute otitis media, in a mammal, such as a dog, cat or human.

In one preferred embodiment of the present invention the apparatus is adapted for the treatment of acute otitis media in an animal and comprises an electromagnetic radiation source capable of providing broad-spectrum electromagnetic radiation, wherein the broad-spectrum electromagnetic radiation has wavelengths of from about 190 nm to about 1200 nm, the broad-spectrum electromagnetic radiation having an intensity sufficient to achieve meaningful suppression in acute otitis media and minimizes erythema on the tympanic membrane of the animal; wherein at least part of the apparatus is adapted for placement proximate to the tympanic membrane of said animal.

2) Method

In accordance with a second aspect of the present invention, a method for achieving the meaningful suppression of the growth potential of a pathogen in a living organism is provided. Also provided within the scope of the third aspect of the present invention is a method for aiding the immune response of a living organism to a pathogen by temporarily suppressing the pathogen. Furthermore, also provided within the scope of the present invention is a method for stimulating the immune system of an organism.

Various apparatus may be employed to practice these methods. Some illustrative apparatus designed to provide short duration pulsed incoherent polychromatic light in a broad-spectrum are described, for example, in U.S. Pat. No. 5,034,235 (Dunn et al.), U.S. Pat. No. 5,489,442 (Dunn et al.), U.S. Pat. No. 5,768,853 (Bushnell et al.) and U.S. Pat. No. 5,786,598 (Clark et al.). Especially preferred for practicing the method of the present invention are the apparatus of the present invention discussed hereinbefore in section 1) above.

In one embodiment of the present invention the method for achieving the meaningful suppression of the growth potential of a pathogen in a living organism comprises applying a broad-spectrum electromagnetic radiation from an apparatus according to the apparatus as described in section 1) above to the living organism at the locus of the pathogen in said living organism.

In one preferred embodiment of the method of the present invention the broad-spectrum electromagnetic radiation is applied to the locus of a pathogen in a living organism is a pulsed broad-spectrum electromagnetic radiation.

In one preferred embodiment the method of the present invention additionally comprises the step of identifying the organism prior to application of the broad-spectrum electromagnetic radiation.

In a preferred embodiment the method of the present invention additionally comprises the step of identifying the pathogen or pathogens prior to application of the broad-spectrum electromagnetic radiation. Typically, the pathogen may be identified by any usual method.

In one preferred embodiment the method of the present invention additionally comprises the step of identifying the locus of the pathogen. Typically, the locus of the pathogen may be identified by any usual method.

In one preferred embodiment the method of the present invention additionally comprises the step of selecting at least one of frequency, duration and intensity of the broad-spectrum electromagnetic radiation. It is especially preferred that this selection of frequency, duration and intensity, is made after the pathogen and/or locus of the pathogen has been identified.

In one preferred embodiment the method of the present invention the intensity of the broad-spectrum electromagnetic radiation is selected to minimize acute tissue effects in the organism at the locus of the pathogen.

In one preferred embodiment the method of the present invention the intensity the broad-spectrum electromagnetic radiation is selected to minimize chronic effects in the organism at the locus of the pathogen.

In preferred embodiment the method of the present invention, comprise the steps of: identifying the living organism, identifying the locus of the pathogen, and selecting the intensity of the broad-spectrum electromagnetic radiation.

In preferred embodiment the method of the present invention, comprise the steps of: identifying the living organism, identifying the pathogen, and selecting at least one of frequency, duration and intensity of the broad-spectrum electromagnetic radiation.

In preferred embodiment the method of the present invention, comprise the steps of: identifying the living organism, identifying the locus of the pathogen, identifying the pathogen, and selecting at least one of frequency, duration and intensity of the broad-spectrum electromagnetic radiation.

The following examples are illustrative of the present invention, but are not meant to limit or otherwise define its scope.

### EXAMPLES

### Example 1

A hand held apparatus for the treatment of the ear containing a housing, which holds power supply, controller, on/off switch, and a bulb which is the electromagnetic radiation source. The apparatus also contains a tip or speculum for facilitating the application of the pulsed broad-spectrum electromagnetic radiation, in this case pathogens located in the ear. Optionally, the device may be fitted with a microprocessor to allow the operator greater flexibility of treatment. The apparatus may also optionally contain a data link to enable a third party to program the treatment regimen form the apparatus or to actually activate the device. The device can be used on humans or other primates with similar auditory systems. The device may also be used on other mammals such as dogs and cats.

### Example 2

This is an apparatus is identical to that of Example 1 except that is contains as the electromagnetic radiation source, two bulbs.

### Example 3

An apparatus that includes a table, on to which the organism is placed, also contains a housing, which holds power supply, controller, on/off switch, and a bulb, which is the electromagnetic radiation source. The apparatus also contains a microprocessor and data link. The apparatus also included interchangeable devices for facilitating the application of the broad-spectrum electromagnetic radiation. For example the apparatus can supply the broad-spectrum electromagnetic radiation through an endoscopic like device for interior applications or via a fibre optical cable for application to the surface of an organism.

### Example 4

An enclosed box, vase or container into which cut flowers are placed contains power supply, controller, on/off switch, and a bulb, which is the electromagnetic radiation source. The device can be preprogrammed by the manufacture to either treat the portion of the flowers at and near the cut of the stems when new cut flowers are placed in the vase, or on a preprogrammed time schedule depending upon the flowers in the vase.

What is claimed is:

1. An apparatus for the meaningful suppression of the growth potential of a pathogen in-vivo, said apparatus comprising an electromagnetic radiation source capable of providing broad-spectrum electromagnetic radiation, wherein said broad-spectrum electromagnetic radiation includes wavelengths of from about 190 nm to about 1200 nm, said broad-spectrum electromagnetic radiation having an intensity sufficient to achieve meaningful suppression in said growth potential of said pathogen in-vivo and wherein at least part of said apparatus is adapted for placement proximate to the in-vivo location of said pathogen.

2. The apparatus according to claim 1 wherein said electromagnetic radiation is a pulsed broad-spectrum electromagnetic radiation and said electromagnetic radiation is pulsed from about 1 to about 1000 times and for a duration of each pulse from about 1 microsecond to about 500 milliseconds.

3. The apparatus according to claim 1 wherein said electromagnetic radiation source is selected from the group consisting of halogen lamps, xenon lamps, halogen enhanced UV lamps, xenon flash lamps, mercury xenon lamps, deuterium lamps, vacuum UV lamps, mercury lamps, lasers and combinations thereof.

4. The apparatus according to claim 1 wherein said broad-spectrum electromagnetic radiation is a continuous spectrum.

5. The apparatus according to claim 1 wherein said broad-spectrum electromagnetic radiation is a combination of at least two discrete spectra.

6. The apparatus according to claim 1 wherein said apparatus comprises a controller, said controller managing the duration and intensity of said electromagnetic radiation source.

7. The apparatus according to claim 1 wherein said apparatus achieves a meaningful suppression in the growth potential of said pathogen in-vivo by increasing the time for said pathogen to double in population.

8. The apparatus according to claim 1 wherein said apparatus achieves a meaningful suppression in the growth potential of said pathogen in-vivo by reducing the population of said pathogen in-vivo by at least about 1 log.

9. The apparatus according to claim 1 wherein said broad-spectrum electromagnetic radiation has an intensity from about 0.01 J/cm² to about 1 J/cm².

10. A method for achieving the meaningful suppression of the growth potential of a pathogen in a living organism comprising applying a broad-spectrum electromagnetic radiation from an apparatus according to claim 1 to said living organism at the locus of said pathogen in said living organism

11. An apparatus for the treatment of acute otitis media in an animal comprising an electromagnetic radiation source capable of providing broad-spectrum electromagnetic radiation, wherein said broad-spectrum electromagnetic radiation has wavelengths of from about 190 nm to about 1200 nm, said broad-spectrum electromagnetic radiation having an intensity sufficient to achieve meaningful suppression in acute otitis media while minimizing erythema on the tympanic membrane of said animal; wherein at least part of said apparatus is adapted for placement proximate to said tympanic membrane of said animal.

12. The apparatus according to claim 11 wherein said electromagnetic radiation is a pulsed broad-spectrum electromagnetic radiation and said electromagnetic radiation is pulsed from about 1 to about 1000 times and for a duration of each pulse from about 1 microsecond to about 500 milliseconds.

13. The apparatus according to claim 11 wherein said apparatus achieves a meaningful suppression in the growth potential of said acute otitis media by increasing the time for the pathogen causing said acute otitis media to double in population.

14. The apparatus according to claim 11 wherein said apparatus achieves a meaningful suppression in the growth potential of said acute otitis media by reducing the population of the pathogen causing said acute otitis media by at least about 1 log.

15. A method for achieving the meaningful suppression of the growth potential of a pathogen in a living organism, said method comprising administering a broad-spectrum electromagnetic radiation to said living organism to locus of said pathogen in said living organism, wherein said broad-spectrum electromagnetic radiation has wavelengths of from about 190 nm to about 1200 nm, said broad-spectrum electromagnetic radiation having an intensity sufficient to achieve meaningful suppression in the growth potential of said pathogen in-vivo.

16. The method according to claim 15 wherein said broad-spectrum electromagnetic radiation is a pulsed broad-spectrum electromagnetic radiation and said electromagnetic radiation is pulsed from about 1 to about 1000 times and for a duration of each pulse from about 1 microsecond to about 500 milliseconds.

17. The method according to claim 15 wherein said pathogen is selected from the group consisting of viruses, bacteria, pyrogens, toxins, fungi, protozoa, prions and combinations thereof.

18. The method according to claim 15 wherein said living organism is an animal.

19. The method according to claim 15 wherein said method achieves a meaningful suppression in the growth potential of said pathogen in-vivo by increasing the time for said pathogen to double in population.

20. The method according to claim 15 wherein said method achieves a meaningful suppression in the growth potential of said pathogen in-vivo by reducing the population of said pathogen in-vivo by at least about 1 log.
